(19) United States
(12) Patent Application Publication
(10) Pub. No.: US 2009/0318741 A1
(43) Pub. Date: Dec. 24, 2009

(54) METHOD OF IMPROVING A DEHYDROGENATION PROCESS

(76) Inventor: Richard Douglas NEWMAN, Houston, TX (US)

Correspondence Address: SHELL OIL COMPANY, P O BOX 2463, HOUSTON, TX, 772522463, US

(21) Appl. No.: 12/419,372

(22) Filed: Apr. 7, 2009

Related U.S. Application Data
(60) Provisional application No. 61/043,419, filed on Apr. 9, 2008.

(51) Int. Cl. C07C 5/32

(52) U.S. Cl. 585/440

(57) ABSTRACT

The invention relates to a method of improving a dehydrogenation process comprising: removing a volume of a first dehydrogenation catalyst from a radial dehydrogenation reactor; loading the reactor with a volume of a second dehydrogenation catalyst that has a lower decline rate than the first dehydrogenation catalyst; and passing a dehydrogenatable hydrocarbon through the reactor wherein the volume of the second catalyst is at most 90% of the volume of the removed catalyst.

This application claims the benefit of U.S. Provisional Application No. 61/043,419, filed Apr. 9, 2008, which is hereby incorporated by reference.

### FIELD OF THE INVENTION

The invention relates to a method of improving a dehydrogenation process.

### BACKGROUND

There have been continuous efforts to improve the efficiency of dehydrogenation process systems. One of the important objectives is ensuring the catalyst is removed after an optimal run length. If it is removed before the optimal run length then the catalyst will have remaining activity that is not used. If the catalyst is removed after the optimal run length then the system is operated at a less than optimal conversion for some time. In most instances, the run length is determined by external factors, so the objective becomes maximizing the use of the catalyst during that run length.

U.S. Patent Application Publication 2002/0065442 describes a method for improving a dehydrogenation process system performance. It discloses the use of an annular, ring-shaped, vertical catalyst bed comprising active catalyst material, contained within a first, ring-shaped vertical layer of the catalyst bed and an inert material, contained within a second, ring-shaped vertical layer of the catalyst bed. The publication discloses that the thickness of the catalyst bed is preferably from 4 to 36 inches, preferably from 6 to 24 inches, most preferably about 18 inches which is thinner than typical dehydrogenation catalyst beds that have a thickness of from 18 inches to 48 inches. The dehydrogenation catalyst used is any conventional commercial or proprietary dehydrogenation catalyst.

The publication discloses that the use of thin catalyst beds reduces the pressure drop across the catalyst bed. The thin beds contain less catalyst, which results in decreased production of the catalyst bed. In addition, the catalyst deactivates over time and operating a reactor with less catalyst results in a decreased run length.

It would be advantageous to provide a method of improving a dehydrogenation system that achieves a reduction in pressure drop across the catalyst bed but avoids a decreased run length or decreased catalyst performance.

### SUMMARY OF THE INVENTION

The invention provides a method of improving a dehydrogenation process comprising: removing a volume of a first dehydrogenation catalyst from a radial dehydrogenation reactor; loading the reactor with a volume of a second dehydrogenation catalyst that has a lower decline rate than the first dehydrogenation catalyst; and passing a dehydrogenatable hydrocarbon through the reactor wherein the volume of the second catalyst is at most 90% of the volume of the removed catalyst.

The invention also provides a method comprising replacing a portion of a dehydrogenation catalyst having a decline rate of from 0.1 to 0.8° C./month in a dehydrogenation reactor with an inert material and introducing a feed comprising a dehydrogenatable hydrocarbon into the reactor wherein the feed entering the reactor contacts the catalyst before contacting the inert material and the pressure drop across the inert material is less than the pressure drop across the replaced portion of dehydrogenation catalyst.

The invention further provides a method of dehydrogenation of a dehydrogenatable hydrocarbon in a radial reactor where a first catalyst was previously removed and a second catalyst was loaded wherein the second catalyst has a lower decline rate than the first catalyst and the volume of the second catalyst is at most 90% of the volume of the first catalyst

### BRIEF DESCRIPTION OF THE DRAWINGS

FIG. 1 depicts a radial reactor that can be used in a dehydrogenation reaction.

FIG. 2 depicts the performance of two alkylaromatic dehydrogenation systems that are operated at a molar steam-to-oil ratio of 9.

FIG. 3 depicts the performance of two alkylaromatic dehydrogenation systems that are operated at a molar steam-to-oil ratio of 7.

FIG. 4 depicts a radial reactor that contains a layer of catalyst and a layer of inert material.

### DETAILED DESCRIPTION OF THE INVENTION

A process for the dehydrogenation of a dehydrogenatable hydrocarbon typically comprises contacting a dehydrogenatable hydrocarbon and steam with a dehydrogenation catalyst to produce the corresponding dehydrogenated hydrocarbon.

The dehydrogenated hydrocarbon formed by the dehydrogenation process is a compound having the general formula:

R¹R²C═CH₂

wherein R¹ and R² independently represent an alkyl, alkenyl or a phenyl group or a hydrogen atom.

The dehydrogenatable hydrocarbon is a compound having the general formula:

R¹R²HC—CH₃

wherein R¹ and R² independently represent an alkyl, alkenyl or a phenyl group or a hydrogen atom.

A suitable phenyl group may have one or more methyl groups as substitutes. A suitable alkyl group generally has from 2 to 20 carbon atoms per molecule, and preferably from 3 to 8 carbon atoms such as in the case of n-butane and 2-methylbutane. Suitable alkyl substituents are propyl (—CH₂—CH₂—CH₃), 2-propyl (i.e., 1-methylethyl, —CH(—CH₃)₂), butyl (—CH₂—CH₂—CH₂—CH₃), 2-methyl-propyl (—CH₂—CH(—CH₃)₂), and hexyl (—CH₂—CH₂—CH₂—CH₂—CH₂—CH₃), in particular ethyl (—CH₂—CH₃). A suitable alkenyl group generally has from about 4 to about 20 carbon atoms per molecule, and preferably from 4 to 8 carbon atoms per molecule.

The dehydrogenatable hydrocarbon may be an alkyl substituted benzene, although other aromatic compounds may be applied as well, such as alkyl substituted naphthalene, anthracene, or pyridine. Examples of suitable dehydrogenatable hydrocarbons are butyl-benzene, hexylbenzene, (2-methylpropyl)benzene, (1-methylethyl)benzene (i.e., cumene), 1-ethyl-2-methyl-benzene, 1,4-diethylbenzene, ethylbenzene, 1-butene, 2-methylbutane and 3-methyl-1-butene. It is possible to convert n-butane with the present process via 1-butene into 1,3-butadiene and 2-methylbutane via tertiary amylenes into isoprene.

Examples of dehydrogenated hydrocarbons that can be produced by the process are butadiene, alpha-methyl styrene, divinylbenzene, isoprene and styrene.

The dehydrogenation process is frequently a gas phase process; wherein a gaseous feed comprising the reactants is contacted with the solid catalyst. The catalyst may be present in the form of a fluidized bed of catalyst particles or in the form of a packed bed. The process may be carried out as a batch process or as a continuous process. Hydrogen may be a further product of the dehydrogenation process, and the dehydrogenation in question may be a non-oxidative dehydrogenation. Examples of applicable methods for carrying out the dehydrogenation process can be found in U.S. Pat. No. 5,689,023; U.S. Pat. No. 5,171,914; U.S. Pat. No. 5,190,906; U.S. Pat. No. 6,191,065, and EP 1027928, which are herein incorporated by reference.

It is advantageous to apply water, which may be in the form of steam, as an additional component of the feed. The presence of water will decrease the rate of deposition of coke on the catalyst during the dehydrogenation process. Typically the molar ratio of water to the dehydrogenatable hydrocarbon in the feed is in the range of from 1 to 50, more typically from 3 to 30, for example from 5 to 10.

The dehydrogenation process is typically carried out at a temperature in the range of from 500 to 700° C., more typically from 550 to 650° C., for example from 600° C. to 640° C. In one embodiment, the dehydrogenation process is carried out isothermally. In other embodiments, the dehydrogenation process is carried out in an adiabatic manner, in which case the temperatures mentioned are reactor inlet temperatures, and as the dehydrogenation progresses the temperature may decrease typically by up to 150° C., more typically by from 10 to 120° C. The absolute pressure is typically in the range of from 10 to 300 kPa, more typically from 20 to 200 kPa, for example 50 kPa, or 120 kPa.

If desired, one, two, or more reactors, for example three or four, may be used. The reactors may be operated in series or parallel. They may or may not be operated independently from each other, and each reactor may be operated under the same conditions or under different conditions.

When operating the dehydrogenation process as a gas phase process using a packed bed reactor, the LHSV may preferably be in the range of from 0.01 to 10 h⁻¹, more preferably in the range of from 0.1 to 2 h⁻¹. As used herein, the term “LHSV” means the Liquid Hourly Space Velocity, which is defined as the liquid volumetric flow rate of the hydrocarbon feed, measured at normal conditions (i.e., 0° C. and 1 bar absolute), divided by the volume of the catalyst bed, or by the total volume of the catalyst beds if there are two or more catalyst beds.

The conditions of the dehydrogenation process may be selected such that the conversion of the dehydrogenatable hydrocarbon is in the range of from 20 to 100 mole %, preferably from 30 to 80 mole %, or more preferably from 35 to 75 mole %.

The activity (T65) of the catalyst is defined as the temperature under given operating conditions at which the conversion of the dehydrogenatable hydrocarbon in a dehydrogenation process is 65 mole %. A more active catalyst thus has a lower T65 than a less active catalyst. The corresponding selectivity (S65) is defined as the selectivity to the desired product at the temperature at which conversion is 65 mole %.

The dehydrogenated hydrocarbon may be recovered from the product of the dehydrogenation process by any known means. For example, the dehydrogenation process may include fractional distillation or reactive distillation. If desirable, the dehydrogenation process may include a hydrogenation step in which at least a portion of the product is subjected to hydrogenation, by which at least a portion of any byproducts formed during dehydrogenation are converted into the dehydrogenated hydrocarbon. The portion of the product subjected to hydrogenation may be a portion of the product that is enriched in the byproducts. Such hydrogenation is known in the art. For example, the methods known from U.S. Pat. No. 5,504,268; U.S. Pat. No. 5,156,816; and U.S. Pat. No. 4,822,936, which are incorporated herein by reference, are readily applicable to the present invention.

One preferred embodiment of a dehydrogenation process is the nonoxidative dehydrogenation of ethylbenzene to form styrene. This embodiment generally comprises feeding a feed comprising ethylbenzene and steam to a reaction zone containing catalyst at a temperature of from about 500° C. to about 700° C. Steam is generally present in the feed at a steam to hydrocarbon molar ratio of from about 7 to about 15. In the alternative this process may be carried out at a lower steam to hydrocarbon molar ratio of from about 1 to about 7, preferably from about 2 to about 6. This process typically produces small amounts of byproducts, for example phenylacetylene and alpha-methyl styrene, in addition to styrene. Alpha-methyl styrene is an undesired byproduct because it acts as a chain terminator when the styrene is later polymerized.

Another preferred embodiment of a dehydrogenation process is the oxidative dehydrogenation of ethylbenzene to form styrene. This embodiment generally comprises feeding ethylbenzene and an oxidant, for example, oxygen, iodide, sulfur, sulfur dioxide, or carbon dioxide to a reaction zone containing catalyst at a temperature of from about 500° C. to about 800° C. The oxidative dehydrogenation reaction is exothermic so the reaction can be carried out at lower temperatures and/or lower steam to oil ratios.

Another preferred embodiment of a dehydrogenation process is the dehydrogenation of isoamylenes to form isoprene. This embodiment generally comprises feeding a mixed isoamylene feed comprising 2-methyl-1-butene, 2-methyl-2-butene, and 3-methyl-1-butene into a reaction zone containing catalyst at a temperature of from about 525° C. to about 675° C. The process is typically conducted at atmospheric pressure. Steam is generally added to the feed at a steam to hydrocarbon molar ratio of from about 13 to about 31.

Another preferred embodiment of a dehydrogenation process is the dehydrogenation of butene to form butadiene. This embodiment generally comprises feeding a mixed butylenes feed comprising 1-butene and 2-butene (cis and/or trans isomers) to a reaction zone containing catalyst at a temperature of from about 500° C. to about 700° C.

Due to the endothermic nature of most of these dehydrogenation processes, additional heat input is often desirable to maintain the required temperatures to maintain conversion and selectivity. The heat can be added before a reaction zone, between reaction zones when there are two or more zones, or directly to the reaction zone.

A preferred embodiment of a suitable heating method is the use of a conventional heat exchanger. The process stream may be heated before entering the first or any subsequent reactors. Preferred sources of heat include steam and other heated process streams.

Another preferred embodiment of a suitable heating method is the use of a flameless distributed combustion heater system as described in U.S. Pat. No. 7,025,940, which is herein incorporated by reference.

Another preferred embodiment of a suitable heating method is catalytic or noncatalytic oxidative reheat. Embodiments of this type of heating method are described in U.S. Pat. No. 4,914,249; U.S. Pat. No. 4,812,597; and U.S. Pat. No. 4,717,779; which are herein incorporated by reference.

The dehydrogenated hydrocarbon produced by the dehydrogenation process may be used as a monomer in polymerization processes and copolymerization processes. For example, the styrene obtained may be used in the production of polystyrene and styrene/diene rubbers. The improved catalyst performance achieved by this invention with a lower cost catalyst leads to a more attractive process for the production of the dehydrogenated hydrocarbon and consequently to a more attractive process which comprises producing the dehydrogenated hydrocarbon and the subsequent use of the dehydrogenated hydrocarbon in the manufacture of polymers and copolymers which comprise monomer units of the dehydrogenated hydrocarbon.

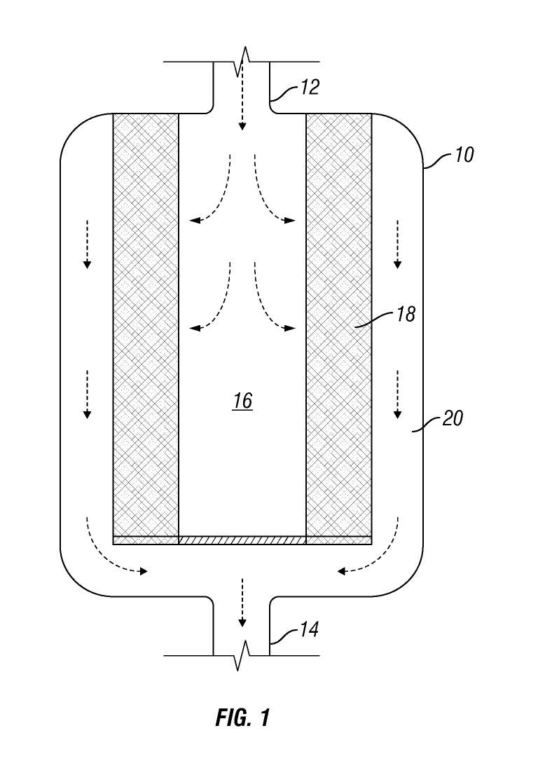

The dehydrogenation reactor for carrying out this dehydrogenation process may be a radial reactor as depicted in FIG. 1. The dehydrogenation reactor 10 is typically situated vertically with the feed inlet 12 at the top or the bottom and the product outlet 14 at the opposite end from the feed inlet. FIG. 1 depicts a downflow reactor with the feed inlet 12 at the top and the product outlet 14 at the bottom, but it may also be operated as an upflow reactor.

The reactor 10 comprises three zones: a feed zone 16, a reaction zone 18, and an effluent zone 20. The feed enters through feed inlet 12 into feed zone 16. The feed then passes through the reaction zone 18, which comprises a dehydrogenation catalyst. The products of the dehydrogenation reaction are then passed into the effluent zone 20 and then removed from the reactor via the product outlet 14.

The reaction zone 18 comprises one or more dehydrogenation catalysts. In addition, reaction zone 18 may comprise one or more inert components that will be described in more detail hereinafter.

The arrows depicted in FIG. 1 show the flow of reactants from the middle of the reactor (feed zone 16) towards the sides of the reactor (effluent zone 20). In the alternative, the reactor may be operated such that the reactants flow from the sides of the reactor through the reaction zone 18 and into the middle of the reactor. In this configuration, zone 16 becomes the effluent zone and zone 20 becomes the feed zone.

The dehydrogenation catalyst may be any suitable catalyst composition that provides for the dehydrogenation of hydrocarbons. A preferred example of a dehydrogenation catalyst composition comprises iron oxide, such as the iron oxide-based dehydrogenation catalysts used in the dehydrogenation of ethylbenzene to yield styrene. The catalyst may additionally comprise potassium oxide.

The iron oxide of the iron oxide based dehydrogenation catalyst may be in a variety of forms including any one or more of the iron oxides, such as, for example, yellow iron oxide (goethite, FeOOH), black iron oxide (magnetite, Fe₃O₄), and red iron oxide (hematite, Fe₂O₃), including synthetic hematite or regenerated iron oxide, or it may be combined with potassium oxide to form potassium ferrite (K₂Fe₂O₄), or it may be combined with potassium oxide to form one or more of the phases containing both iron and potassium as represented by the formula (K2O)ₓ.(Fe₂O₃)_{y}.

Typical iron oxide based dehydrogenation catalysts comprise from 10 to 90 weight percent iron, calculated as Fe₂O₃, and up to 40 weight percent potassium, calculated as K₂O. The iron oxide based dehydrogenation catalyst may further comprise one or more promoter metals that are usually in the form of an oxide. These promoter metals may be selected from the group consisting of Sc, Y, Ti, Zr, Hf, V, Nb, Ta, Cr, Mn, Tc, Re, Ru, Os, Co, Rh, Ir, Ni, Pd, Pt, Cu, Au, Zn, Cd, Hg, Al, Ga, In, Tl, Si, Ge, Sn, Pb, P, As, Sb, Bi, S, Se, Te and mixtures of any two or more thereof. Among the promoter metals, preferred are those selected from the group consisting of Ca, Mg, Mo, W, Ce, La, Cu, Cr, V and mixtures of two or more thereof. Most preferred are Ca, Mg, W, Mo, and Ce.

A preferred iron oxide based dehydrogenation catalyst comprises from 40 to 90 weight percent iron, calculated as Fe₂O₃, from 5 to 30 weight percent potassium, calculated as K₂O; from 2 to 20 weight percent cerium, calculated as Ce₂O₃; from 1 to 10 weight percent molybdenum, calculated as MoO₃; and from 1 to 10 weight percent an alkaline earth metal, calculated as an oxide.

Descriptions of typical iron oxide-based dehydrogenation catalysts that are used as dehydrogenation catalysts may be found in patent publications that include U.S. Patent Publication No. 2003/0144566 A1; U.S. Pat. No. 5,689,023; U.S. Pat. No. 5,376,613; U.S. Pat. No. 4,804,799; U.S. Pat. No. 4,758,543; U.S. Pat. No. 6,551,958 B1; and EP 0,794,004 B1, all of such patent publications are incorporated herein by reference.

The catalyst may be prepared by any method known to those skilled in the art. For example, a paste may be formed comprising iron oxide, alkali metal or a compound thereof, silver or a compound thereof and any additional catalyst component(s). A mixture of these catalyst components may be mulled and/or kneaded or a homogenous or heterogeneous solution of any of these components may be impregnated on the iron oxide. Sufficient quantities of each component may be calculated from the composition of the catalyst to be prepared. Examples of applicable methods can be found in U.S. Pat. No. 5,668,075; U.S. Pat. No. 5,962,757; U.S. Pat. No. 5,689,023; U.S. Pat. No. 5,171,914; U.S. Pat. No. 5,190,906, U.S. Pat. No. 6,191,065, and EP 1027928, which are herein incorporated by reference.

The catalyst components may be shaped into pellets of any suitable form, for example, tablets, spheres, pills, saddles, trilobes, twisted trilobes, tetralobes, rings, stars, hollow and solid cylinders, and asymmetrically lobed particles as described in U.S. Patent Application Publication 2005/0232853. The addition of a suitable quantity of water, for example up to 30 wt %, typically from 2 to 20 wt %, calculated on the weight of the mixture, may facilitate the shaping into pellets. If water is added, it may be at least partly removed prior to calcination. Suitable shaping methods are pelletizing, extrusion, and pressing. Instead of pelletizing, extrusion or pressing, the mixture may be sprayed or spray dried to form a catalyst. If desired, spray drying may be extended to include pelletization and calcination.

An additional compound may be combined with the mixture that acts as an aid to the process of shaping and/or extruding the catalyst, for example a saturated or unsaturated fatty acid (such as palmitic acid, stearic acid, or oleic acid) or a salt thereof, a polysaccharide derived acid or a salt thereof, or graphite, starch, or cellulose. Any salt of a fatty acid or polysaccharide derived acid may be applied, for example an ammonium salt or a salt of any metal mentioned hereinbefore. The fatty acid may comprise in its molecular structure from 6 to 30 carbon atoms (inclusive), preferably from 10 to 25 carbon atoms (inclusive). When a fatty acid or polysaccharide derived acid is used, it may combine with a metal salt applied in preparing the catalyst, to form a salt of the fatty acid or polysaccharide derived acid. A suitable quantity of the additional compound is, for example, up to 1 wt %, in particular 0.001 to 0.5 wt %, relative to the weight of the mixture.

After formation, the catalyst mixture may be dried and calcined. Drying generally comprises heating the catalyst at a temperature of from about 30° C. to about 500° C., preferably from about 100° C. to about 300° C. Drying times are generally from about 2 minutes to 5 hours, preferably from about 5 minutes to about 1 hour. Calcination generally comprises heating the catalyst, typically in an inert, for example nitrogen or helium or an oxidizing atmosphere, for example an oxygen containing gas, air, oxygen enriched air or an oxygen/inert gas mixture. The calcination temperature is typically at least about 600° C., or preferably at least about 700° C., more preferably at least 825° C., and most preferably at least 880° C. The calcination temperature will typically be at most about 1600° C., or preferably at most about 1300° C. Typically, the duration of calcination is from 5 minutes to 12 hours, more typically from 10 minutes to 6 hours.

Low decline rate catalysts have been developed and are disclosed in US Patent Application Publication 2006-0106269, which is herein incorporated by reference. A “stable” or “low decline rate” dehydrogenation catalyst is defined as a catalyst that exhibits a decline rate that averages at most 0.7° C./month when used under certain specified standard reaction conditions. The decline rate preferably averages at most 0.6° C./month and more preferably at most 0.5° C./month.

The decline rate is defined as the amount the average reactor inlet temperature must be increased to hold conversion constant and is expressed in degrees Centigrade per month. This temperature increase to maintain conversion is required due to the deactivation of the catalyst.

The stable or low decline rate dehydrogenation catalysts are distinguished from other dehydrogenation catalysts principally by their low decline rate rather than by their composition. Their low decline rate characteristics in comparison to the other dehydrogenation catalysts, however, may be, but are not required to be, due to compositional differences.

To determine the decline rate, the temperature of the feed mixture introduced into a dehydrogenation reactor is adjusted to provide a conversion of the dehydrogenatable hydrocarbon of 65 percent. The decline rate is determined by the average increase in the feed mixture temperature necessary to maintain a constant conversion of 65 percent during the time period. The decline rate is expressed as the change in T(65) per change in time (1 month), (e.g., ΔT(65)/Δtime), or ° C./month.

Conventional dehydrogenation catalysts are not considered to be of the type having a low decline rate and will not exhibit the characteristics of the low decline rate dehydrogenation catalysts. These catalysts will exhibit decline rates that are higher than those of the low decline rate catalysts. It is understood that a higher decline rate means that the catalyst will tend to deactivate with use at a greater rate than will a catalyst having a lower decline rate, thus, being less stable. Therefore, conventional dehydrogenation catalysts exhibit decline rates averaging greater than 0.7° C./month, but more typically, their decline rate averages at least 0.75° C./month, and, most typically, the decline rate averages at least 0.8° C./month.

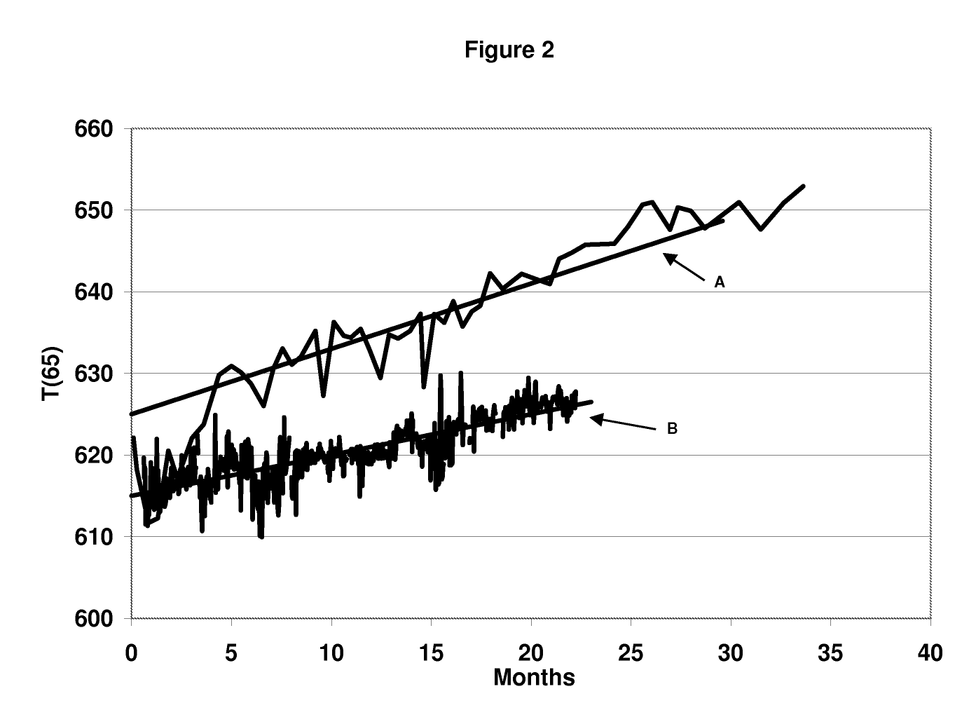

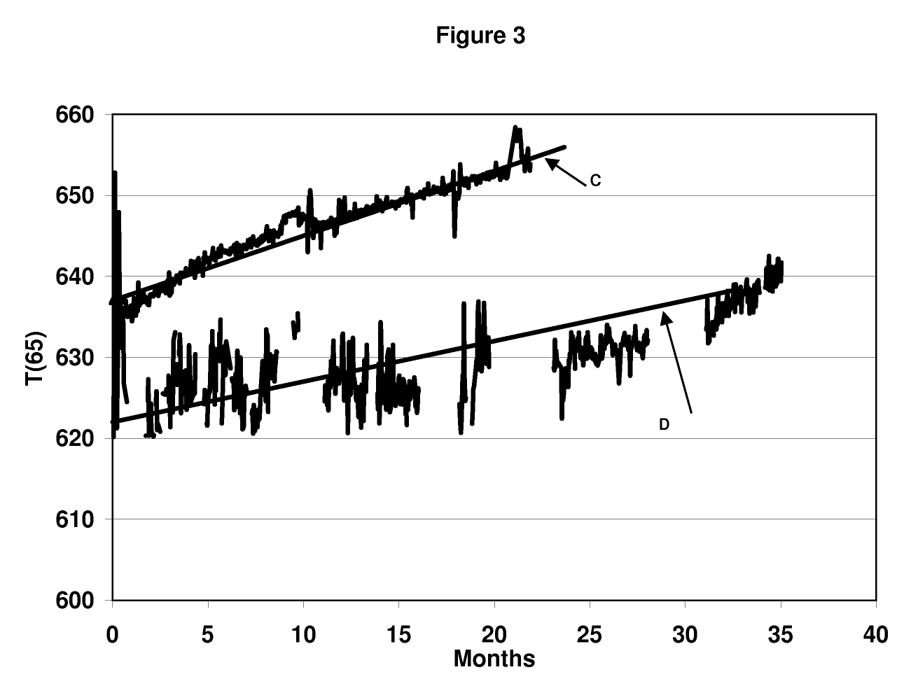

FIGS. 2 and 3 depict the decline rate of two different catalysts employed in ethylbenzene dehydrogenation reactors: one a low decline rate catalyst and the other a conventional catalyst. The data in the figures is from commercial runs of the catalyst and are normalized to average plant conditions. FIG. 2 depicts the catalyst decline rate for two catalysts: B, a low decline rate catalyst, and A, a conventional catalyst, operated at a steam-to-oil molar ratio of 9, an LHSV of 0.45 hr⁻¹, an ethylbenzene conversion of 65%, and an average system pressure of 9 psia. FIG. 3 depicts the catalyst decline rate for two catalysts: D, a low decline rate catalyst, and C, a conventional catalyst operated at a steam-to-oil molar ratio of 7, an LHSV of 0.45 hr⁻¹, an ethylbenzene conversion of 65%, and an average system pressure of 9 psia.

The decline rate of the catalyst is an important feature of the catalyst because it determines how long the reactor can be operated before it needs to be shutdown for a catalyst replacement. As the catalyst deactivates, the reactor inlet temperature is increased to maintain a constant conversion. At a time hereinafter referred to as end of run, the temperature cannot be increased further due to safety and/or economic considerations. For typical ethylbenzene dehydrogenation process systems, the end of run usually occurs when the reactor inlet temperature reaches 650° C.

Run lengths can be from 1 month to 72 months and possibly even longer. Reactors loaded with conventional catalyst typically have run lengths of 6 months to 36 months, and more typically 12 months to 24 months. During catalyst replacement, the reactor is shutdown so it is clear that there is an economic advantage to extending the run length of the reactor if possible. Often the run length cannot be extended due to external factors such as required plant maintenance so another possibility is to maintain the run length, but use less catalyst.

The low decline rate catalysts can be used to improve the operation of a dehydrogenation process system. In one embodiment, some or all of the conventional catalyst is removed from a dehydrogenation reactor, and the reactor is loaded with a low decline rate catalyst. As will be described hereinafter, the volume of low decline rate catalyst loaded is less than the volume of conventional catalyst that was removed.

First, the conventional catalyst is removed from the dehydrogenation reactor. This catalyst may be partly or completely deactivated. Then, the reactor is loaded with a low decline rate catalyst. The volume of low decline rate catalyst that is loaded is less than the volume of conventional catalyst that is removed in the first step. The volume of the low decline rate catalyst may be at most 90% of the volume of the conventional catalyst, preferably at most 75%, and more preferably at most 50% of the volume of the conventional catalyst that was removed.

Due to the qualities of the low decline rate catalyst, less catalyst is required than when the reactor is loaded with conventional catalyst. When using an existing reactor, this results in empty space in the catalyst bed. The catalyst is preferably loaded so that it is present from the top to the bottom of the catalyst bed. This results in a catalyst bed that has two annular layers, one with catalyst and another that may be empty or filled with any inert material known to one skilled in the art. The catalyst may be held in place by a screen or other device. The inert material is anything that does not have a significant impact on the dehydrogenation reaction, either by promoting or inhibiting the reaction or catalyst performance.

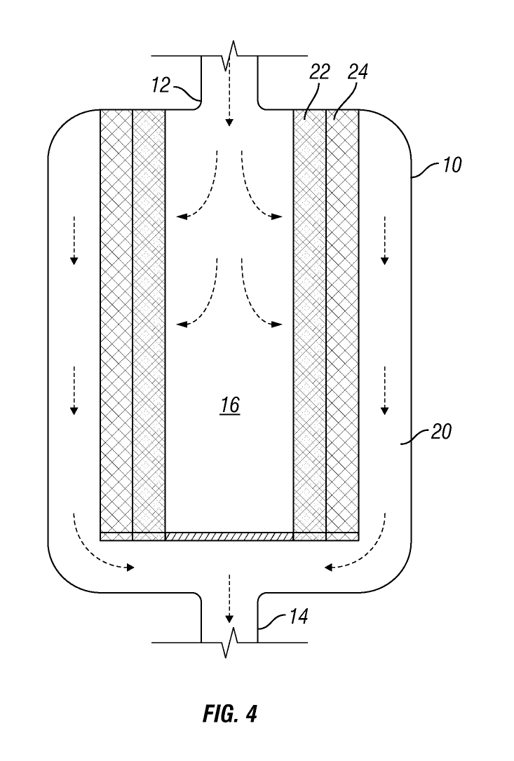

FIG. 4 depicts an embodiment of the reactor that contains low decline rate catalyst and an inert material. The reactor is similar to the reactor depicted in FIG. 1, but the reaction zone 18 is divided into two zones: a catalyst zone 22 and an inert zone 24.

The inert material is preferably a material with a larger diameter than the catalyst present in the reactor that results in reduced pressure drop across the catalyst bed. A reduced pressure drop results even if the inert material only has a slightly larger diameter than the catalyst.

The decreased pressure drop across the reactor allows the reactor to be operated at a lower average pressure that results in an increased selectivity to styrene in an ethylbenzene dehydrogenation reaction system. If the volume of low decline rate catalyst is 75% of the volume of the conventional catalyst being replaced then the selectivity achieved may be about 0.3% higher. If the volume of low decline rate catalyst is 50% of the volume of the conventional catalyst being replaced then the selectivity achieved may be about 0.5% higher.

The feed entering the reactor may contact either the inert or the catalyst first, but it is preferred for the feed to contact the catalyst first. If the feed contacts the inert first, then undesirable thermal reactions can occur since the feed is at the dehydrogenation reaction temperature. Since the dehydrogenation reaction is typically endothermic, the feed will preferably contact the catalyst first, resulting in a decrease in temperature before the feed contacts the inert material.

In addition, the method of the invention may be applied to a reactor that has been loaded with a full load of low decline rate catalyst. Such a reactor usually finishes its run with remaining activity that is wasted when the catalyst is removed. It is more economical to load the catalyst bed with a reduced volume of catalyst so that the catalyst can be more fully used. This will be described in more detail through the use of hypothetical examples that follow.

The following hypothetical examples are presented to illustrate the invention, but they should not be construed as limiting the scope of the invention.

### Comparative Example 1

This comparative example describes the operation of a hypothetical conventional ethylbenzene dehydrogenation catalyst in a radial reactor with a decline rate of 0.9° C./month as depicted in FIG. 2 for catalyst A. The catalyst is operated at a steam-to-oil molar ratio of 9, an LHSV of 0.45 hr⁻¹, an ethylbenzene molar conversion of 65%, and an average system pressure of 9 psia. This example assumes a maximum reactor inlet temperature of 650° C. The reactor catalyst bed is filled with conventional catalyst. The reactor inlet temperature needed to provide a conversion of 65% was calculated at 3 months and 24 months. The data is shown in Table 1, and it shows that the reactor reaches end of run conditions at 24 months.

### Comparative Example 2

This example calculates the inlet temperature of a radial reactor at 3 months and at 24 months to maintain a constant 65% conversion for a reactor as in Example 1 wherein the total volume of conventional catalyst is removed and the reactor is filled with a hypothetical low decline rate catalyst as depicted in FIG. 2 as catalyst B. The catalyst had a decline rate of 0.6° C./month. All process variables besides temperature were the same as in Example 1. The results are shown in Table 1 and show that there is still activity remaining in the catalyst at 24 months.

### Example 3

This example calculates the inlet temperature of the reactor at 3 months and at 24 months to maintain a constant 65% conversion for the reactor of Example 1 wherein the total volume of conventional catalyst is removed and the reactor is 75% filled with the same low decline rate catalyst described in Example 2. That means that the volume of catalyst in this reactor is 75% of the volume of catalyst used in Examples 1 and 2. All process variables besides temperature were the same as in Example 1. The results are shown in Table 1 and show that there is still some remaining catalyst activity at 24 months.

### Example 4

This example calculates the inlet temperature of the reactor at 3 months and at 24 months to maintain a constant 65% conversion for the reactor of Example 1 wherein the total volume of conventional catalyst is removed and the reactor is 50% filled with the same low decline rate catalyst described in Example 2. All process variables besides temperature were the same as in Example 1. The results are shown in Table 1 and show that the reactor has reached end of run conditions.

As can be seen from these examples, it is possible to achieve the same run length of 24 months using a reactor full of conventional catalyst or a reactor 50% full with low decline rate catalyst. Alternatively, the reactors in Examples 2 and 3 could be operated for longer than 24 month run lengths before the inlet temperature reached the maximum of 650° C. The higher inlet temperatures at 3 months for Examples 3 and 4 are required to compensate for higher liquid hourly space velocity.

TABLE 1

| | Decline | Catalyst volume | Inlet Temp. | Inlet Temp |
|---|---|---|---|---|
| Ex. | rate | (% of Ex. 1 volume) | at 3 months | at 24 months |
| 1 | 0.9 | — | 631 | 650 |
| 2 | 0.5 | 100% | 631 | 642 |
| 3 | 0.5 | 75% | 635 | 646 |
| 4 | 0.5 | 50% | 639 | 650 |

What is claimed is:

1. A method of improving a dehydrogenation process comprising:
removing a volume of a first dehydrogenation catalyst from a radial dehydrogenation reactor;
loading the reactor with a volume of a second dehydrogenation catalyst that has a lower decline rate than the first dehydrogenation catalyst; and
passing a dehydrogenatable hydrocarbon through the reactor
wherein the volume of the second catalyst is at most 90% of the volume of the removed catalyst.

2. A method as claimed in claim 1 wherein the volume of second catalyst is at most 75% of the volume of the removed catalyst.

3. A method as claimed in claim 1 wherein the volume of second catalyst is at most 50% of the volume of the removed catalyst.

4. A method as claimed in claim 1 wherein an inert material is loaded into the reactor.

5. A method as claimed in claim 3 wherein the dehydrogenatable hydrocarbon contacts the catalyst before contacting the inert material.

6. A method as claimed in claim 3 wherein the pressure drop across the inert material is less than the pressure drop across the catalyst that was removed.

7. A method as claimed in claim 1 wherein the decline rate of the first catalyst is at least 1.1 times the decline rate of the second catalyst.

8. A method as claimed in claim 1 wherein the decline rate of the first catalyst is at least 1.5 times the decline rate of the second catalyst.

9. A method as claimed in claim 1 wherein the decline rate of the first catalyst is at least 1.8 times the decline rate of the second catalyst.

10. The method as claimed in claim 1 wherein the activity of the second catalyst decreases at a rate of from 0.1 to 0.8° C./month.

11. The method as claimed in claim 1 wherein the activity of the second catalyst decreases at a rate of from 0.4 to 0.6° C./month.

12. The method as claimed in claim 1 wherein the activity of the first catalyst decreases at a rate greater than 0.8° C./month.

13. The method as claimed in claim 1 wherein the dehydrogenation process is an alkylaromatic dehydrogenation process.

14. The method as claimed in claim 1 wherein the dehydrogenation process is an ethylbenzene dehydrogenation process.

15. A method comprising replacing a portion of a dehydrogenation catalyst having a decline rate of from 0.1 to 0.8° C./month in a dehydrogenation reactor with an inert material and introducing a feed comprising a dehydrogenatable hydrocarbon into the reactor wherein the feed entering the reactor contacts the catalyst before contacting the inert material and the pressure drop across the inert material is less than the pressure drop across the replaced portion of dehydrogenation catalyst.

16. The method as claimed in claim 14 wherein the activity of the stable dehydrogenation catalyst decreases at a rate of from 0.4 to 0.6° C./month.

17. A method of dehydrogenation of a dehydrogenatable hydrocarbon in a radial reactor where a first catalyst was previously removed and a second catalyst was loaded wherein the second catalyst has a lower decline rate than the first catalyst and the volume of the second catalyst is at most 90% of the volume of the first catalyst.
